(19) United States
(12) Patent Application Publication
(10) Pub. No.: US 2017/0340663 A1
(43) Pub. Date: Nov. 30, 2017

(54) MINERAL COMPLEX FORMULATIONS FOR ORAL ADMINISTRATION AND METHODS FOR THEIR USE

(71) Applicant: Justin Cannock, Stuart, FL (US)

(72) Inventor: Justin Cannock, Stuart, FL (US)

(73) Assignee: AVELIS LLC, Gretna, LA (US)

(21) Appl. No.: 15/626,339

(22) Filed: Jun. 19, 2017

Related U.S. Application Data
(63) Continuation of application No. PCT/US2015/066694, filed on Dec. 18, 2015.
(60) Provisional application No. 62/094,628, filed on Dec. 19, 2014.

(51) Int. Cl. A61K 33/08; A61K 9/48

(52) U.S. Cl. CPC A61K 33/08; A61K 9/4841; A61K 2300/00

(57) ABSTRACT

The invention provides an oral formulation for dietary supplementation comprising a binder and a mineral complex consisting essentially of a natural basaltic deposit having an average particle size ranging from about 10 to about 6000 mesh and comprising about 40 wt. % to about 60 wt. % Si0₂, about 6 wt. % to about 16 wt. % Fe₂0₃, about 4 wt. % to about 12 wt. %. CaO, and about 2 wt. % to about 8 wt. % MgO. Methods of formulating and using the aforesaid oral formulation also are provided.

### CROSS-REFERENCE TO RELATED APPLICATIONS

This application claims the benefit of U.S. Provisional Patent Application No. 62/094,628, which was filed on Dec. 19, 2014, and which is incorporated by reference in its entirety herein.

### BACKGROUND OF THE INVENTION

The present invention relates generally to dietary supplements. More specifically, the present invention relates to dietary supplements for oral administration, and related methods for their use.

### BRIEF SUMMARY OF THE INVENTION

The invention also provides an oral formulation for dietary supplementation comprising a binder and a mineral complex consisting essentially of a natural basaltic deposit having an average particle size ranging from about 10 to about 6000 mesh and comprising about 40 wt. % to about 60 wt. % SiO₂, about 6 wt. % to about 16 wt. % Fe₂0₃, about 4 wt. % to about 12 wt. %. CaO, and about 2 wt. % to about 8 wt. % MgO. Other optional ingredients, such as probiotic microorganisms and/or vitamins, also may be included in the formulations.

In another aspect, the invention provides a method for supplementing the diet of an animal, such as a human, equine, or other domesticated animal, in need thereof comprising administering to the animal the oral formulations as described and claimed herein.

In a related aspect, the oral formulations for dietary supplementation as described and claimed herein are provided as tablet, a dissolvable tablet, a chewable tablet, a free-flowing powder, a capsule, a gelcap or as part of a foodstuff.

The invention also provides a process for preparing a supplemented liquid for oral consumption by an animal comprising: (a) providing a prepared or formulated liquid; and (b) adding to the liquid a palatable amount of the oral formulations as described and claimed herein.

The invention further provides methods for reducing fatigue, reducing exercise time of recovery, lean weight gain, body fat loss, improved sleep quality, improved health of skin/hair/nails, increasing concentration, increasing muscle tone, increasing exercise performance, and/or decreasing joint pain in an animal comprising administering to the animal the inventive compositions in accordance with the regimens described and claimed herein.

### DETAILED DESCRIPTION OF THE INVENTION

The invention provides an oral formulation for dietary supplementation comprising a mineral complex consisting essentially of natural basaltic deposit (rock) that can be administered to animals, including humans, alone or in combination with various other consumables, as well as methods of use.

The mineral complex useful in the context of the present invention may be obtained from basaltic rock. In this regard, the mineral complex of the invention is a natural product which meets Organic Materials Review Institute (OMRI) certification standards as organic/non-synthesized inputs: natural mineral deposits that are not chemically processed/altered. The mineral complex of the invention also qualifies as Generally Recognized As Safe (GRAS) status for food processing applications.

The mineral complex of the invention is obtained from volcanic basaltic (mineral) deposits (rock). For example, the mineral deposit may be located in non-porous naturally altered volcanic lava, from highly porous naturally altered volcanic ash, or the mineral complex may be prepared from a combination of non-porous naturally altered volcanic lava and highly porous naturally altered volcanic ash. The mineral complex is not thermally processed prior to use (e.g., it is not subjected to a combustion process, nor is it the product of a combustion process), but is desirably extracted, milled to the desired size, and formulated for use.

The mineral complex of the invention may be obtained from any source that extracts mineral deposits having the characteristics described herein. The mineral complex may be obtained from one source or from several different sources. For example, the mineral complex may be prepared from one natural source of mineral deposit having the characteristics described herein, or the mineral complex can be prepared by mixing together several different mineral deposits to achieve a mineral complex having the characteristics described herein. It should be further understood that when the mineral complex of the invention is obtained from a natural source, the mineral complex may vary in content from source to source and batch to batch. However, analyses of the mineral complex may be routinely performed in accordance with techniques known to those skilled in the art to ensure that the quality of the mineral complex is maintained from batch to batch and from source to source.

The mineral complex of the invention comprises numerous minerals and elements as described herein. Unless otherwise indicated by language or context, references to weight percents of the minerals and elements of which the mineral complex is comprised are based on the total weight of the mineral complex, and are described herein in terms of the formulation as a whole.

The component having the highest weight percent (wt. %) in the mineral complex is silicon dioxide (SiO₂). For example, the mineral complex may comprise about 28 wt. % to about 50 wt. %, about 30 wt. % to about 48 wt. %, about 32 wt. % to about 46 wt. %, about 34 wt. % to about 44 wt. %, about 36 wt. % to about 40 wt. %, about 37 wt. % to about 39 wt. %, or about 38 wt. % Si0₂. Desirably, the mineral complex comprises less than 40 wt. % (e.g., about 35 wt. %, about 36 wt. %, about 37 wt. %, about 38 wt. % or about 39 wt. %), SiO₂.

The mineral complex of the invention also may comprise an iron oxide (e.g., FeO, Fe₂O₃, or Fe₃O₄). For example, the mineral complex may comprise about 2 wt. % to about 9 wt. % (e.g., about 2 wt. %, about 3 wt. %, about 4 wt. %, about 5 wt. %, about 6 wt. %, about 7 wt. %, about 8 wt. %, or about 9 wt. %), about 3 wt. % to about 8 wt. %, about 4 wt. % to about 7 wt. %, about 5 wt. % to about 6 wt. %, or about 5.1, 5.2, 5.3, 5.4, 5.5, 5.6, 5.7, 5.8 or 5.9 wt. %, Fe₂0₃.

The mineral complex of the invention also may comprise calcium oxide (CaO). For example, the mineral complex may comprise about 2 wt. % to about 9 wt. % (e.g., about 2 wt. %, about 3 wt. %, about 4 wt. %, about 5 wt. %, about 6 wt. %, about 7 wt. %, about 8 wt. %, or about 9 wt. %), about 3 wt. % to about 8 wt. %, about 4 wt. % to about 7 wt. %, about 5 wt. % to about 6 wt. %, or about 5.1, 5.2, 5.3, 5.4, 5.5, 5.6, 5.7, 5.8 or 5.9 wt. %, CaO.

The mineral complex of the invention also may comprise magnesium oxide (MgO). For example, the mineral complex may comprise about 0.5 wt. % to about 5 wt. % (e.g., about 0.5 wt. %, about 1 wt. %, about 1.5 wt. %, about 2 wt. %, about 2.5 wt. %, about 3 wt. %, about 3.5 wt. %, about 4 wt %, about 4.5 wt. %, or about 5 wt. %), about 1 wt. % to about 4.5 wt. %, about 1.5 wt. % to about 4 wt. %, about 2 wt. % to about 3 wt. %, or about 2.1, 2.2, 2.3, 2.4, 2.5, 2.6, 2.7, 2.8 or 2.9 wt. %, MgO.

The mineral complex of the invention also may comprise sodium oxide (Na₂0). For example, the mineral complex may comprise about 0.1 wt. % to about 5 wt. % (e.g., about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, or about 5 wt. %), about 1 wt. % to about 4.5 wt. %, about 1 wt. % to about 4 wt. %, about 1 wt. % to about 3 wt. %, or about 1.0, 1.2, 1.3, 1.4, 1.5, 1.6, 1.7, 1.8 or 1.9 wt. %, Na₂0.

The mineral complex of the invention also may further comprise potassium oxide (K₂O). For example, the mineral complex may comprise about 1 wt. % to about 12 wt. % (e.g., about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, or about 12 wt. %), about 2 wt. % to about 10 wt. %, about 3 wt. % to about 9 wt. %, about 5 wt. % to about 9 wt. %, or about 7.5, 7.6, 7.7, 7.8, 7.9, 8.0, 8.1, 8.2 or 8.3 wt. %, K₂O.

It is contemplated that the foregoing ranges of each component of the mineral complex may be present in the mineral complex in any combination. For example, the mineral complex may comprise the foregoing components in the following ranges (which may be further combined with other components described herein, e.g., rare earths, oxidized components, and those disclosed in Tables 2 and 3):

TABLE 1

| | Ranges | | |
|---|---|---|---|
| Compo- | (weight percent based on total weight of mineral complex) | | |
| nent | General | Desired | Preferred |
| SiO₂ | about 28 to | about 34 to | about 36 to |
| | about 50 | about 44 | about 40 |
| Fe₂0₃ | about 2 to about 9 | about 4 to about 7 | about 5 to about 6 |
| CaO | about 2 to about 9 | about 4 to about 7 | about 5 to about 6 |
| MgO | about 0.5 to about 5 | about 1 to about 4 | about 2 to about 3 |
| Na₂0 | about 0.1 to about 5 | about 0.5 to about 3 | about 1 to about 2 |
| K₂0 | about 1 to about 12 | about 5 to about 10 | about 7 to about 9 |
| P₂0₅ | about 0.5 to about 5 | about 1 to about 4 | about 2 to about 3 |
| Al₂O₃ | about 0.01 to | about 0.01 to | about 0.01 to |
| | about 15 | about 10 | about 9 |

The mineral complex of the invention also may further comprise aluminum oxide (Al₂O₃). Preferably, the mineral complex may comprise less than about 6 wt. % Al₂O₃, about 12 wt. % or about 10 wt. %. For example, the mineral complex may comprise about 0.01 wt. % to about 15 wt. %, about 1 wt. % to about 12 wt. % (e.g., about 1 wt. %, about 2 wt. %, about 3 wt. %, about 4 wt. %, about 5 wt. %, about 6 wt. %, about 7 wt. %, about 8 wt. % about 9 wt. %, about 10 wt. %, about 11 wt. %, or about 12 wt. %) about 1 wt. % to about 10 wt. %, about 1 wt. % to about 9 wt. % Ai0₃, or about 1, 2, 3, 4, 5, 6, 7, 8 or 9 wt. %, Al₂O₃.

Desirably, the mineral complex also may comprise at least one or more rare earth elements (e.g., at least two, at least three, at least four, or at least five). As defined by IUPAC, rare earth elements (which include for purposes of the invention that which may be referred to as rare earth metals) are a collection of seventeen chemical elements in the periodic table, namely scandium (Sc), yttrium (Y), and the fifteen lanthanoids: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). The rare earth elements are also referred to as light rare earth elements (lanthanum, cerium praseodymium, neodymium, promethium, and samarium) and heavy rare earth elements (europium, gadolinium, terbium, dysprosium, holmium, erbium, thulium, ytterbium, and lutetium). Scandium and yttrium are considered rare earths since they tend to occur in the same ore deposits as the lanthanoids and exhibit similar chemical properties.

In this regard, the mineral complex may comprise one or more rare earth elements selected from the group consisting of scandium, yttrium, lanthanum, cerium praseodymium, neodymium, promethium, samarium, europium, gadolinium, terbium, dysprosium, holmium, erbium, thulium, ytterbium, and lutetium. It is desirable that the mineral complex comprise at least one light rare earth element and at least one heavy rare earth element. In other embodiments, the mineral complex may comprise only light rare earth elements or only heavy rare earth elements. In a preferred embodiment, the mineral complex may comprise less than 50 ppm of each rare earth element. For example, the mineral complex may comprise about 40 ppm, about 30 ppm, about 25 ppm, about 20 ppm, about 15 ppm, about 10 ppm, about 5 ppm, about 4 ppm, bout 3 ppm, about 2 ppm, about 1 ppm, or about 0.5 ppm of one or more rare earth elements. In another embodiment the mineral complex may comprise about 0.5 ppm to about 49 ppm, about 0.5 ppm to about 40 ppm, about 0.5 ppm to about 30 ppm, about 0.5 ppm to about 25 ppm, about 0.5 ppm to about 20 ppm, about 0.5 ppm to about 15 ppm, about 0.5 ppm to about 10 ppm, about 0.5 ppm to about 5 ppm, about 0.5 ppm to about 1 ppm, or about 0.5 ppm of one or more rare earth elements.

In a further embodiment, the mineral complex may comprise one or more of the following oxidized components: chromium oxide (Cr₂O₃), titanium oxide (TiO₂), manganese oxide (MnO), phosphorous oxide (P₂O₅), strontium oxide (SrO), and barium oxide (BaO). For example, the mineral complex may comprise about 0.001 wt. % to about 3 wt. %. about 0.01 wt. % to about 3 wt. %, about 0.01 wt. % to about 2 wt. %, about 0.1 wt. % to about 1 wt. %, or about 0.5 wt. % to about 1 wt. % Cr₂O₃, Ti0₂, MnO, P₂O₅, SrO, and/or BaO. In one embodiment, the mineral complex comprises less than about 3 wt. % (e.g., about 2.5 wt. %, about 2 wt. %, about 1.5 wt. %, about 1 wt. %, about 0.5 wt. %, or less than 0.5 wt. %) Cr₂O₃, TiO₂, MnO, P₂O₅, SrO, and/or BaO. In another embodiment, the mineral complex comprises one or more of the following: about 0.01 wt. % to about 0.03 wt. % Cr₂0₃, about 0.1 wt. % to about 1 wt. % Ti0₂, about 0.01 wt. % to about 0.3 wt. % MnO, about 1 wt. % to about 3 wt. % P₂0₅, about 0.01 wt. % to about 0.05 wt. % SrO, and/or about 0.01 wt. % to about 0.1 wt. % BaO.

The mineral complex of the invention also may comprise carbon (C). Preferably, the mineral complex comprises from about 1 to about 20 wt. % C. For example, the mineral complex may comprise about 2 wt. %, about 18 wt. %, about 5 wt. %, about 15 wt. %, about 8 wt. % to about 14 wt. %, about 10 wt. % to about 14 wt. %, about 12 wt. % C, or about 9, 10, 11, 12, 13, or 14 wt. % C. The mineral complex of the invention also may comprise sulfur (S). Preferably, the mineral complex comprises less than 1% S. For example, the mineral complex may comprise about 0.01 wt. % to about 1 wt. %, about 0.02 wt. % to about 0.5 wt. %, about 0.05 wt. % to about 0.1 wt. %, or about 0.01, 0.02, 0.03, 0.04, 0.05, 0.06, 0.07, 0.08, 0.09 or 0.1 wt. %, S.

The mineral complex of the invention also may further comprise at least one or more of (e.g., at least two or more of, at least three or more of, at least four or more of, or at least five or more of) the following: silver (Ag), barium (Ba), boron (B), cobalt (Co), chromium (Cr), caesium (Cs), copper (Cu), gallium (Ga), hafnium (Hf), molybdenum (Mo), niobium (Nb), nickel (Ni), lead (Pb), rubidium (Rb), tin (Sn), strontium (Sr), tantalum (Ta), thorium (Th), thallium (Tl), uranium (U,) vanadium (V), tungsten (W), zinc (Zn), and zirconium (Zr). When present, the mineral complex preferably comprises less than 1000 ppm each of Ag; Ba, Co, Cr, Cs, Cu, Ga, Hf, Mo, Nb, Ni, Pb, Rb, Sn, Sr, Ta, Th, Tl, U, V, W, Zn, or Zr. For example, the mineral complex may comprise one or more of Ag, Ba, Co, Cr, Cs, Cu, Ga, Hf, Mo, Nb, Ni, Pb, Rb, Sn, Sr, Ta, Th, Tl, U, V, W, Zn, and Zr in the ranges set forth in Table 2.

TABLE 2

| | Ranges | | |
|---|---|---|---|
| | (ppm based on total weight of mineral complex) | | |
| Element | General | Desired | Preferred |
| Ag | about 0.0001 ppm to | about 0.0001 ppm to | about 0.0001 ppm to |
| | about 5 ppm | about 2 ppm | about 1 ppm |
| Ba | about 1 ppm to about | about 100 ppm to about | about 300 ppm to about |
| | 1000 ppm | 750 ppm | 550 ppm |
| B | about 1 ppm to about 500 | about 5 ppm to about 100 | about 10 ppm to about 50 |
| | ppm | ppm | ppm |
| Co | about 0.01 ppm to about | about 0.01 ppm to about | about 0.01 ppm to about |
| | 100 ppm | 50 ppm | 20 ppm |
| Cr | about 1 ppm to about | about 1 ppm to about 100 | about 25 ppm to about |
| | 200 ppm | ppm | 100 ppm |
| Cs | about 0.0001 ppm to | about 0.0001 ppm to | about 0.0001 ppm to |
| | about 50 ppm | about 10 ppm | about 5 ppm |
| Cu | about 1 ppm to about | about 1 ppm to about 200 | about 1 ppm to about 100 |
| | 400 ppm | ppm | ppm |
| Ga | about 0.001 ppm to | about 0.001 ppm to about | about 0.001 ppm to about |
| | about 200 ppm | 100 ppm | 20 ppm |
| Hf | about 0.001 ppm to | about 0.001 ppm to about | about 0.001 ppm to about |
| | about 50 ppm | 1 O ppm | 5 ppm |
| Mo | about 0.001 ppm to | about 0.001 ppm to about | about 0.001 ppm to about |
| | about 25 ppm | 5 ppm | 1 ppm |
| Nb | about 0.001 ppm to | about 0.001 ppm to about | about 0.001 ppm to about |
| | about 100 ppm | 90 ppm | 10 ppm |
| Ni | about 1 ppm to about | about 1 ppm to about 100 | about 1 ppm to about 50 |
| | 400 ppm | ppm | ppm |
| Pb | about 0.001 ppm to | about 0.001 ppm to about | about 0.001 ppm to about |
| | about 25 ppm | 15 ppm | 10 ppm |
| Rb | about 1 ppm to about | about 1 ppm to about 200 | about 1 ppm to about 100 |
| | 400 ppm | ppm | ppm |
| Sn | about 0.0001 ppm to | about 0.0001 ppm to | about 0.0001 ppm to |
| | about 15 ppm | about 5 ppm | about 1 ppm |
| Sr | about 1 ppm to about | about 1 ppm to about 400 | about 1 ppm to about 250 |
| | 600 ppm | ppm | ppm |
| Ta | about 0.0001 ppm to | about 0.0001 ppm to | about 0.0001 ppm to |
| | about 10 ppm | about 2 ppm | about ppm |
| Th | about 0.001 ppm to | about 0.001 ppm to about | about 0.001 ppm to about |
| | about 100 ppm | 20 ppm | 10 ppm |
| Tl | about 0.0001 ppm to | about 0.0001 ppm to | about 0.0001 ppm to |
| | about 10 ppm | about 2 ppm | about 1 ppm |
| U | about 0.0001 ppm to | about 0.0001 ppm to | about 0.0001 ppm to |
| | about 25 ppm | about 5 ppm | about 2.5 ppm |
| V | about 1 ppm to about | about 1 ppm to about 200 | about 1 ppm to about 100 |
| | 600 ppm | ppm | ppm |
| W | about 0.0001 ppm to | about 0.0001 ppm to | about 0.0001 ppm to |
| | about 25 ppm | about 200 ppm | about 2.5 ppm |
| Zn | about 1 ppm to about 400 | about 1 ppm to about 200 | about 1 ppm to about 100 |
| | ppm | ppm | ppm |
| Zr | about 1 ppm to about 400 | about 1 ppm to about 200 | about 1 ppm to about 150 |
| | ppm | ppm | ppm |

The mineral complex of the invention also may comprise one or more of (e.g., one, two, three, four, five, or all six of): arsenic (As), bismuth (Bi), mercury (Hg), antimony (Sb), selenium (Se), cadmium (Cd) and tellurium (Te). Preferably, the mineral complex comprises less than 5 ppm of As, less than 1 ppm of Cd, less than 5 ppm of Bi, less than 1 ppm of Hg, less than 5 ppm of Sb, less than 5 ppm of Se, and/or less than 5 ppm of Te. For example, the mineral complex may comprise one or more of As, Cd, Bi, Hg, Sb, Se and Te in the ranges set forth in Table 3.

TABLE 3

| | Ranges | | |
|---|---|---|---|
| | (ppm based on total weight of mineral complex) | | |
| Element | General Range | Desired Range | Preferred Range |
| As | about 0.0001 ppm to | about 0.0001 ppm to | about 0.0001 ppm |
| | about 4 ppm | about 3 ppm | to about 2 ppm |
| Cd | about 0.0001 ppm to | about 0.0001 ppm to | about 0.0001 ppm |
| | about 2 ppm | about 1 ppm | to about 0.5 |
| Bi | about 0.0001 ppm to | about 0.0001 ppm to | about 0.0001 ppm |
| | about 4 ppm | about 3 ppm | to about 2 ppm |
| Hg | about 0.00001 ppm | about 0.00001 ppm | about 0.00001 ppm |
| | to about 0.5 ppm | to about 0.3 ppm | to about 0.2 min |
| Sb | about 0.0001 ppm to | about 0.0001 ppm to | about 0.0001 ppm |
| | about 2 ppm | about 2 ppm | to about 2 ppm |
| Se | about 0.0001 ppm to | about 0.0001 ppm to | about 0.0001 ppm |
| | about 4 ppm | about 2 ppm | to about 1 ppm |
| Te | about 0.0001 ppm to | about 0.0001 ppm to | about 0.0001 ppm |
| | about 2 ppm | about 1 ppm | to about 0.5 ppm |

In one embodiment, the mineral complex described herein has one or more (e.g., at least two, at least three, at least four, or at least five) of the following characteristics: an average weight in tons per cubic yard of about 1 to about 1.5 (e.g., about 1.3); a loss on ignition of about 0.2% to about 0.3% (e.g., about 0.25%); a fusion of about 2100 to about 2300 degrees Fahrenheit (e.g., about 2200); a mill abrasion loss (A.R.E.A.) of about 5% to about 6% (e.g., about 5.4%); a L.A. abrasion loss according to ASTM C535-89 of about 7.2 to about 8.2 (e.g., about 7.7); a L.A. abrasion loss according to ASTM C 131-89 of about 10.3 to about 11.3 (e.g., about 10.8); a specific gravity according to ASTM C97 of about 2.900 to about 3.060 (e.g., about 2.980); a specific gravity according to ASTM C 127 of about 2.900 to about 3.060 (e.g., about 2.980); an absorption according to ASTM C 127 of less than about 0.5% (e.g., less than 0.4%); and/or a soundness loss according to ASTM C 88 Mg Su of about 0.25% to about 0.75% (e.g., about 0.5%).

The mineral complex of the invention may be obtained in any physical size. Preferably, however, the mineral complex is crushed, ground, and/or milled into a powdered form using any routine methods known in the art.

In one embodiment, the mineral complex is in a powdered form wherein the particles have an average particle size of about 10 to about 6000 mesh. In one embodiment, the particles are very fine and have an average particle size of about 400 to about 6000 mesh, which corresponds to a size of about 37 microns to about 1 micron. For example, and in this embodiment, the particles may have an average particle size of about 400 to about 6000 mesh, about 400 to about 5000 mesh, about 400 to about 4000 mesh, about 400 to about 3000 mesh, about 400 to about 2000 mesh, about 400 to about 1000 mesh, about 400 to about 900 mesh, about 400 to about 800 mesh, about 400 to about 700 mesh, about 400 to about 600 mesh, about 400 to about 500 mesh, about 500 to about 6000 mesh, about 600 to about 6000 mesh, about 700 to about 6000 mesh, about 800 to about 6000 mesh, about 900 to about 6000 mesh, about 1000 to about 6000 mesh, about 2000 to about 6000 mesh, about 3000 to about 6000 mesh, about 4000 to about 6000 mesh, about 5000 to about 6000 mesh, about 500 to about 5000 mesh, about 600 to about 4000 mesh, about 700 to about 3000 mesh, about 800 to about 2000 mesh, about 900 to about 1000 mesh, about 1000 to about 6000 mesh, about 1500 to about 5500 mesh, about 2500 to about 5000 mesh, about 3000 to about 4500 mesh, about 3500 to about 4000 mesh, or about 4000 to about 6000 mesh.

In a related embodiment, the particles are slightly larger, having an average particle size of about 200 to about 400 mesh, which corresponds to a size of about 74 microns to about 37 microns. For example, and in this embodiment, the particles may have an average particle size of about 200 to about 400 mesh, about 230 to about 400 mesh, about 250 to about 400 mesh, about 275 to about 400 mesh, about 300 to about 400 mesh, about 325 to about 400 mesh, about 350 to about 400 mesh, about 375 to about 400 mesh, about 200 to about 375 mesh, about 200 to about 350 mesh, about 200 to about 325 mesh, about 200 to about 300 mesh, about 200 to about 275 mesh, about 200 to about 250 mesh, about 200 to about 225 mesh, or about 250 to about 350 mesh.

In yet another related embodiment, the particles have an even larger particle size of about 10 to about 200 mesh, which corresponds to a size of about 2000 microns to about 74 microns. For example, and in this embodiment, the particles can have an average particle size of about 10 to about 200 mesh, about 20 to about 200 mesh, about 40 to about 200 mesh, about 60 to about 200 mesh, about 80 to about 200 mesh, about 100 to about 200 mesh, about 120 to about 200 mesh, about 140 to about 200 mesh, about 160 to about 200 mesh, about 180 to about 200 mesh, about 10 to about 180 mesh, about 10 to about 160 mesh, about 10 to about 140 mesh, about 10 to about 120 mesh, about 10 to about 100 mesh, about 10 to about 80 mesh, about 10 to about 60 mesh, about 10 to about 40 mesh, about 10 to about 20 mesh, about 40 to about 180 mesh, about 60 to about 160 mesh, or about 80 to about 140 mesh. In a further related embodiment, the particles have an average particle size of about 50 to about 400 mesh, about 100 to about 400 mesh, about 120 to about 400 mesh, about 140 to about 400 mesh, or about 170 to about 400 mesh. The conversion of mesh sizes to microns is well known in the art.

The mineral complex described herein is useful in a variety of different applications, as described herein. Upon referring to the disclosure provided herein, one skilled in the art will appreciate that the particle size of the mineral complex may be selected based on the type of application in which the mineral complex is being used. In addition, one skilled in the art upon reading this disclosure should also appreciate that mineral complex having a relatively smaller average particle size (e.g., those described previously as being a powdered form) distributes/suspends/dissolves more readily in oral formulations and in the gut after ingestion.

In addition, one skilled in the art should also appreciate that in some embodiments of the invention it is desirable to use a mineral complex comprising a variety of different average particle sizes. In this regard, it is contemplated that the mineral complex may comprise any combination of the foregoing ranges of particle sizes. For example, the mineral complex (and compositions thereof) described herein may comprise a certain amount of mineral complex having an average particle size of about 400 to about 6000 mesh, and/or a further amount of mineral complex having an average particle size of about 200 to about 400 mesh, and/or an additional amount of mineral complex having an average particle size of about 10 to about 200 mesh. By way of further example, the mineral complex (and compositions thereof) may comprise about 5% to about 85% (e.g., about 5%, about 10%, about 15%, about 20%, about 25%, about 30%, about 35%, about 40%, about 45%, about 50%, about 55%, about 60%, about 65%, about 70%, about 75%, about 80%, or about 85%) of the mineral complex having an average particle size of about 400 to about 6000 mesh, and/or about 15% to about 95% (e.g., about 15%, about 20%, about 25%, about 30%, about 35%, about 40%, about 45%, about 50%, about 55%, about 60%, about 65%, about 70%, about 75%, about 80%, about 85%, about 90%, or about 95%) of the mineral complex having an average particle size of about 200 to about 400 mesh, and/or about 5% to about 75% (e.g., about 5%, about 10%, about 15%, about 20%, about 25%, about 30%, about 35%, about 40%, about 45%, about 50%, about 55%, about 60%, about 65%, about 70%, or about 75%) of the mineral complex having an average particle size of about 10 to about 200 mesh. For example, the mineral complex (and compositions thereof) described herein may comprise about 50% of mineral complex having an average particle size of about 400 to about 6000 mesh and about 50% of mineral complex having an average particle size of about 200 to about 400 mesh, or the composition may comprise about 30% of mineral complex having an average particle size of about 400 to about 6000 mesh, about 50% of mineral complex having an average particle size of about 200 to about 400 mesh, and about 20% of mineral complex having an average particle size of about 10 to about 200 mesh.

Although average particle size may be conveniently measured by scanning electron microscopy (SEM) in accordance with techniques known to those skilled in the art, other techniques also may be used. Preferably, at least 75%, at least 80%, at least 85%, at least 90%, at least 95%, at least 96%, at least 97%, at least 98%, or at least 99% of the particles have a particle size falling within the ranges described herein.

The oral formulations and methods for use contemplate that the oral formulations are provided in a form suitable for oral administration, e.g., a tablet, a dissolvable tablet, a chewable tablet, a free-flowing powder, a capsule, a gelcap, or incorporated into a food (e.g., biscuit, treat, kibble or gummy). When formulated into a tablet, dissolvable tablet, chewable tablet, capsule or gelcap, a variety of optional ingredients may be used, e.g., carriers, binders, flavor agents, sweeteners. When formulated as a free-flowing powder, the formulation may be added directly to a liquid, such as water, milk, an energy drink, soda, juice, or other beverage of choice, mixed thoroughly, and orally administered or ingested. Methods for preparing these formulations are well known to those skilled in the art.

The binder as used in the context of the present invention may constitute one or more of a variety of components, which may be a synthetic or desirably an organic component, and which provides one or more advantageous properties to the inventive formulation. For example, some of the binders may be useful in assisting in the agglomeration of the formulation components into a tablet or chewable tablet (as known in the art), while others may have the benefit of augmenting the properties of the oral formulation more generally. These components may include brewers condensed soluble, wheat powder, beet syrup, molasses, such as beet molasses, desugared beet molasses, or cane molasses, honey, humic substances, whey, starch, gelatin, sodium casein, sulfur, wax, polymer, oil, urea-formaldehyde, plant starches, protein gels, glues, gumming compositions, seaweed, peat, crystallizing compounds, gelling clays, synthetic gel-forming compounds, and mixtures thereof. Additional examples of binders that may be used herein include carbohydrates, such as monosaccharides, disaccharides, oligosaccharides, and polysaccharides; proteins; lipids; glycolipid; glycoprotein; lipoprotein; and combinations and derivatives of the same. Exemplary carbohydrates include glucose, mannose, fructose, galactose, sucrose, lactose, maltose, xylose, arabinose, trehalose, and mixtures thereof, such as corn syrup; celluloses, such as carboxymethylcellulose (CMC), ethylcellulose, hydroxyethylcellulose, hydroxy-methylethylcellulose, hydroxyethylpropylcellulose, methylhydroxyethyl-cellulose, and methylcellulose; starches, such as amylose, seagel, alpha-starch, carboxy-alpha-starch, starch acetates, starch hydroxyethyl ethers, ionic starches, long-chain alkyl starches, dextrins, amine starches, phosphates starches, and dialdehyde starches; plant starches, such as corn starch and potato starch; other carbohydrates, such as pectin, amylopectin, xylan, glycogen, agar, alginic acid, phycocolloids, chitin, gum arabic, guar gum, gum karaya, gum tragacanth, and locust bean gum; complex organic substances, such as lignin and nitrolignin; derivatives of lignin, such as lignin sulfonate and lignosulfonate salts, including calcium lignosulfonate and sodium lignosulfonate; and complex carbohydrate-based compositions containing organic and inorganic ingredients such as molasses. Suitable protein binders include, for example, soy extract, zein, protamine, collagen, and casein. Binders operative herein also include synthetic organic polymers, such as oxide polymers, polyacrylamides, polyacrylates, polyvinyl pyrrolidone, polyethylene glycol, polyvinyl alcohol, polyvinylmethyl ether, polyvinyl acrylates, and polylactic acid.

The binder may be present in the composition from about 0.1 wt. % to about 99.5 wt. % of the oral composition (e.g., about 0.1 wt. %, about 0.5 wt. %, about 1 wt. %, about 2 wt. %, about 3 wt. %, about 4 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 96 wt. %, about 97 wt. %, about 98 wt. %, about 99 wt. %, or about 99.5 wt. % binder). Desirably, the composition comprises from about 0.1 wt. % to about 50 wt. % binder (e.g., from about 0.1 wt. % to about 5 wt. %, from about 0.5 wt. % to about 10 wt. %, from about 1 wt. % to about 20 wt. %, from about 5 wt. % to about 25 wt. %, or from about 10 wt. % to about 50 wt. % binder). More desirably, the composition comprises from about 0.5 wt. % to about 2.5 wt. % binder (e.g., from about 0.5 wt. % to about 1 wt. %, from about 0.5 wt. % to about 1.5 wt. %, from about 0.5 wt. % to about 2 wt. %, from about 1 wt. % to about 2.5 wt. %, from about 1.5 wt. % to about 2.5 wt. %, from about 2 wt. % to about 2.5 wt. %, or from about 1 wt. % to about 2 wt. %).

The invention also provides methods of using the mineral complex described herein. These methods comprise administering to an animal, e.g., a human, the mineral complex/formulations comprising the mineral complex, as described herein.

For example, the mineral complex may be administered to a human at any beneficial dosage, but generally a dosage regimen of about 0.001 to about 100 grams/70 kg body weight/day may be used, desirably from about 0.01 to about 75 grams/70 kg body weight/day, more desirably from about 0.25 to about 50 grams/70 kg body weight/day, and even more desirably from about 0.3 to about 30 grams/70 kg body weight/day, and most desirably from about 0.5 to about 20 grams/70 kg body weight/day, with total daily dosages of from about 100 to 2000 mg, 200 to 1200 mg, 300 to 1000 mg, 600 to 1000 mg, as well as 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg and 2000 mg, each per 70 kg/body weight/day, also being contemplated by the invention. The administration may be as a single dose, or divided into multiple doses, e.g., twice or three-times daily. Desirably, the mineral complex is administered with food, e.g., the formulation may be mixed into or eaten with food (which includes drink).

For example, the administration to equine may range from about 0.5 g/day to about 250 g/day; desirably from about 1 g/day to about 150 g/day; and preferably from about 2 g/day to about 100 g/day. For domesticated animals, such as dogs and cats, the administration may range from about 5 mg/day to about 5000 mg/day; desirably from about 10 mg/day to about 3000 mg/day; and preferably from about 50 mg/day to about 2500 mg/day.

The invention further contemplates combining the mineral complex formulations as described herein with one or more of other commercially-available supplements, such as creatine, glutamine, nitric oxide, probiotics, vitamins and amino acids, to name but a few.

The invention also contemplates the addition of one or more probiotic microorganisms, wherein the microorganisms may be one or more of the following genera: Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcm, or Lactobacillus. Desirably, the probiotic comprises one or more of the following: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enlerococcusfdecalis, Lactobacillus bulgaricus Lactobacillus acidophilus, Lactobacillus alimentarius, Lactobacillus casei subsp. casei, Lactobacillus casei Shirola, Lactobacillus curvatus, Lactobacillus delbruckii subsp. lactis, Lactobacillus far ciminus, Lactobacillus gasseri, Lactobacillus helve ticus, Lactobacillus johnsonii, Lactobacillus reuteri, Lactobacillus rhamnosus (Lactobacillus GG), Lactobacillus sake, Lactobacillus sporogenes, Lactococcus lactis, Micrococcus various, Pediococcus acidilactici, Pediococcus pentosaceus, Pediococcus acidilactici, Pediococcus halophilus, Streptococcus faecalis, Streptococcus thermophiius, Streptococcus faecium, Staphylococcus carnosus, Leuconosioc meseMeroki.es ssp cremoris, Pediococcus acidolactici, Pediococcus cerevisiae, Bifidobacterium bifidus; Bifidobacterium, longum, Brevibacterium linens, Propiombacterium shermanii, Propiombacterium arabinosum, Penicillhtm roquefortii, PeniciUium camemhertii or Staphylococcus xylosus.

The other supplements may be taken in accordance with their respective labels with the subject mineral complex formulations on a daily basis.

While not desiring to be bound to a particular theory, it is believed that the inventive formulations provide additional oxygen to a subject by means other than via the respiratory system. This is desirable because non-respiratory passive diffusion of oxygen is required, for example, to maintain sight. By way of further example, the largest organ of the body, the skin, has its outermost layer supplied almost exclusively by atmospheric oxygen. Similarly, blood oxygen does not supply the oxygen requirements of digestive microbes; these microbes may access oxygen via the degradation of ingested nutrients, if sufficient oxygen is contained within the nutrients consumed. However, the oxygen requirements of digestive microbes can outstrip the gastrointestinal availability of oxygen. Thus, it is believed that the oxygen-containing elements in the inventive formulations serve to allow the introduction of oxygen into the animal via non-respiratory means, and in a relatively high percentage (rather than oxygen as a liquid or gas). As shown herein, the oxygen is present in both simple and complex bonded minerals, and released during digestion as both atomic and molecular oxygen (as an allotrope of elemental oxygen, O2), and thus provides this needed non-respiratory oxygen to the animal.

While not desiring to be bound to a particular theory, it is believed that the present invention provides an additional source of oxygen, i.e., a source of oxygen provided other than by respiration.

The mineral complex formulations of the present invention provide a variety of benefits when administered to humans, including reduced fatigue, increased stamina, accelerates recovery from injury or surgery, reduction in exercise time of recovery, lean weight gain, body fat loss, increases blood O₂ carrying capacity (increase in hemoglobin), increase in the oxygenation of tissues, improved sleep quality, improved health of skin/hair/nails, increased concentration, increased muscle tone, increased exercise performance, increased oxygenation, improved digestion, improves insulin sensitivity, increased nutrient absorption, and decreased joint pain. After administration to equines, the formulation has been reported to increase blood O₂ carrying capacity, increase oxygenation of tissues, reduced insulin sensitivity, increased oxygenation, increased nutrient absorption, and bolster overall health and performance.

The following examples further illustrate the invention but, of course, should not be construed as in any way limiting its scope.

### Example 1

An 8-week trial with a 44 year old human male subject was conducted. The male subject was monitored for 8 weeks prior to the trial. The pre-trial diet was maintained at an average daily intake of 2000 calories, with an exercise regimen of 45 minutes per day, broken into 20 minutes cardio via a treadmill, and 25 minutes resistance via a ‘universal’ cable-type machine. Over the 8 week pre-trial period the subject's body weight fluctuated between 231 lbs and 254 lbs. As the subject's weight was relatively stable given the low caloric intake, the anticipated resting metabolic rate (RMR) was less than 2000 calories. The subject was rated for daily activity level, with a reading of ‘Low Active’ and a total energy expenditure (TEE) of RMR+800 calories per day, giving a TEE of <2600.

During the 8 week trial the subject maintained a 2000 calorie per day intake and exercised for 45 minutes per day. Between 1 pm and 3 pm each day the subject consumed a single size 00 gelatin capsule (600 mg) of a mineral complex having the composition as described in the preferred columns of Tables 2 and 3 and as follows: about 38 wt. % Si0₂, about 9 wt. % Al₂O₃, about 5 wt. % Fe₂O₃, about 5 wt. % CaO, about 2 wt. % MgO, about 2 wt. % Na₂0, about 8 wt. % K₂O, about 3 wt. % P₂0₅, about 12 wt. % C, about 0.1 wt. % S, about 0.01 wt % Cr₂O₃, about 1 wt. % Ti0₂, about 0.1 wt. % MnO, about 0.02 wt. % SrO, and about 0.1 wt. % BaO.

At week 1 (start), weeks 2, 4, 6 and week 8 (finish) the subject was reviewed. Lab results indicate a consistent and rapid body mass increase despite a low caloric intake. Most notably, body composition results showed that the body mass increase of 20.5 lbs consisted of 20.25 lbs of fat free mass (muscle, bone, organs, water). Fat mass gain was 0.251 b (¼ of 1.0 lb). Body fat percentage decreased 2.1%. Body volume increased 8.47 liters. Thoracic gas volume remained unchanged at 4.46 liters.

The subject also began the trial with long-standing significant joint pain of the left shoulder, this pain restricting the range of motion and weight that could be lifted, and actively interrupted the subject's sleep. After 2 weeks on the formulation the joint pain had reduced by approximately 80%, and sleep interruption was reduced.

### Example 2

A subject with a 20-year old foot injury (broken foot) experienced pain on rising in the morning, when the first 10 minutes or so of putting weight on the foot would produce significant pain. After 7 to 10 days of a single daily capsule of the formulation (as described in Example 1,600 mg) the subject reported a dramatic reduction and cessation of pain in the foot. 8 weeks later the subject stopped the trial, and within 2 weeks the foot pain had returned, though of a lesser severity. He then began a daily dose of the formulation, and within 7 days the pain had ceased.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-20. (canceled)

21. An oral formulation for dietary supplementation comprising about 10 wt. % to about 50 wt. % binder and a mineral complex consisting essentially of a natural basaltic deposit having an average particle size ranging from about 10 to about 6000 mesh and comprising about 38 wt. % Si0₂, about 9 wt. % Al₂O₃, about 5 wt. % Fe₂O₃, about 5 wt. % CaO, about 2 wt. % MgO, about 2 wt. % Na20, about 8 wt. % K₂O, about 3 wt. % P₂0₅, about 12 wt. % C, about 0.1 wt. % S, about 0.01 wt % Cr₂O₃, about 1 wt. % Ti0₂, about 0.1 wt. % MnO, about 0.02 wt. % SrO, and about 0.1 wt. % BaO.

22. The oral formulation according to claim 21, wherein the oral formulation is a tablet, dissolving tablet, a chewable tablet, capsule, free-flowing powder or gelcap.

23. The oral formulation according to claim 22, wherein the formulation is provided as a free-flowing powder within a capsule.

24. The oral formulation according to claim 21, wherein the oral formulation is incorporated into a foodstuff.

25. The oral formulation according to claim 21, wherein the oral formulation is a liquid.

26. The oral formulation according to claim 21, wherein the binder is one of more of: brewers condensed solubles; wheat powder; beet syrup; molasses; honey; humic substances; whey; gelatin; sodium casein; wax; digestible oil; glues; gumming compositions; seaweed; peat; crystallizing compounds; gelling clays; or synthetic gel-forming compounds.

27. The oral formulation according to claim 21, wherein the binder is one or more of: saccharide; protein; lipid; glycolipid; glycoprotein; glucose; mannose; fructose; galactose; sucrose; lactose; maltose; xylose; arabinose; trehalose; corn syrup; cellulose; starch; plant starches; pectins; xylan; glycogen; agar; alginic acid; phycocolloids; chitin; gums; lignin; oxide polymers; polyacrylamides; polyacrylates; polyvinyl pyrrolidone; polyethylene glycol; polyvinyl alcohol; polyvinylmethyl ether; polyvinyl acrylate; or polylactic acid.

28. The oral formulation according to claim 21, wherein the formulation further comprises at least one microorganism comprising one or more of the following genera: Bifidobacterium, Bacteroides. Clostridium, Fusobacterium, Melissococcus. Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcm, or Lactobacillus.

29. The oral formulation according to claim 28, wherein the at least one microorganism is selected from the group consisting of: Saccharomyces cereviseae. Bacillus coagulans, Bacillus licheniformis, Bacillus subtilis, Bifidobacterium bifidmn. Bifidobacterium infantis. Bifidobacterium longum, Enterococcus faecium, Enter ococcusfaecalis. Lactobacillus bulgaricus, Lactobacillus; acidophilus, Lactobacillus alimentarius. Lactobacillus casci subsp. casei, Lactobacillus casei Shirota. Lactobacillus curvatus, Lactobacillus delbruckii subsp. lactis, Lactobacillus farciminus, Lactobacillus gusseri, Lactobacillus helveticus, Lactobacillus johnsonii. Lactobacillus reuteri, Lactobacillus rhamnosus (Lactobacillus GG), Lactobacillus sake, Lactobacillus sporogenes, Lactococcus lactis. Micrococcus varians. Pediococcus acidilactici, Pediococcus peniosaceus. Pediococcus acidilactici, Pediococcus halophilus, Streptococcus faecalis, Streptococcus thermophilus, Streptococcus faecium, Staphylococcus carnosus. Leuconostoc mesenteroides ssp cretnoris, Pediococcus acidolactici, Pediococcus cerevisiae, Bifidobacterium bifidus; Bifidobacterium longum, Brevibacterium linens, Propionibacterium shermanii; Propionibacterium arabinosum. Penicillium roquefortii, Penicillium camembertii and Staphylococcus xylosus.

30. A method for supplementing the diet of an animal in need thereof comprising orally administering to the animal the formulation according to claim 21 in a regimen of about 0.001 grams to about 100 grams/70 kg body weight/day.

31. The method according to claim 29, wherein the formulation is administered in a regimen of about 0.25 grams to about 50 grams/70 kg body weight/day.

32. An oral formulation for dietary supplementation comprising about 10 wt. % to about 50 wt. % humic and a mineral complex consisting essentially of a natural basaltic deposit having an average particle size ranging from about 10 to about 6000 mesh and comprising about 38 wt. % Si02, about 9 wt. % Al2O3, about 5 wt. % Fe2O3, about 5 wt. % CaO, about 2 wt. % MgO, about 2 wt. % Na20, about 8 wt. % K2O, about 3 wt. % P205, about 12 wt. % C, about 0.1 wt. % S, about 0.01 wt % Cr2O3, about 1 wt. % Ti02, about 0.1 wt. % MnO, about 0.02 wt. % SrO, about 0.1 wt. % BaO and

| Element | Amount |
|---|---|
| Ag | about 0.0001 ppm to about 1 ppm |
| Ba | about 300 ppm to about 550 ppm |
| B | about 10 ppm to about 50 ppm |
| Co | about 0.01 ppm to about 20 ppm |
| Cr | about 25 ppm to about 100 ppm |
| Cs | about 0.0001 ppm to about 5 ppm |
| Cu | about 1 ppm to about 100 ppm |
| Ga | about 0.001 ppm to about 20 ppm |
| Hf | about 0.001 ppm to about .5 ppm |
| Mo | about 0.001 ppm to about 1 ppm |
| Nb | about 0.001 ppm to about 10 ppm |
| Ni | about 1 ppm to about 50 ppm |
| Pb | about 0.001 ppm to about 10 ppm |
| Rb | about 1 ppm to about 100 ppm |
| Sn | about 0.0001 ppm to about 1 ppm |
| Sr | about 1 ppm to about 250 ppm |
| Ta | about 0.0001 ppm to about 1 ppm |
| Th | about 0.001 ppm to about 10 ppm |
| Tl | about 0.0001 ppm to about 1 ppm |
| U | about 0.0001 ppm to about 2.5 ppm |
| V | about 1 ppm to about 100 ppm |
| W | about 0.0001 ppm to about 2.5 ppm |
| Zn | about 1 ppm to about 100 ppm |
| Zr | about 1 ppm to about 150 ppm |

33. The oral formulation according to claim 32, wherein the oral formulation is a tablet, dissolving tablet, a chewable tablet, capsule, free-flowing powder or gelcap.

34. The oral formulation according to claim 33, wherein the formulation is provided as a free-flowing powder within a capsule.

35. The oral formulation according to claim 32, wherein the oral formulation is incorporated into a foodstuff.

36. The oral formulation according to claim 32, wherein the oral formulation is a liquid.

37. The oral formulation according to claim 32, wherein the formulation further comprises at least one microorganism selected from the group consisting of: Saccharomyces cereviseae. Bacillus coagulans, Bacillus licheniformis, Bacillus subtilis, Bifidobacterium bifidmn. Bifidobacterium infantis. Bifidobacterium longum, Enterococcus faecium, Enter ococcusfaecalis. Lactobacillus bulgaricus, Lactobacillus; acidophilus, Lactobacillus alimentarius. Lactobacillus casei subsp. casei, Lactobacillus casei Shirota. Lactobacillus curvatus, Lactobacillus delbruckii subsp. lactis, Lactobacillus farciminus, Lactobacillus gusseri, Lactobacillus helveticus, Lactobacillus johnsonii. Lactobacillus reuteri, Lactobacillus rhamnosus (Lactobacillus GG), Lactobacillus sake, Lactobacillus sporogenes, Lactococcus lactis. Micrococcus varians. Pediococcus acidilactici, Pediococcus peniosaceus. Pediococcus acidilactici, Pediococcus halophilus, Streptococcus faecalis, Streptococcus thermophilus, Streptococcus faecium, Staphylococcus carnosus. Leuconostoc mesenteroides ssp cretnoris, Pediococcus acidolactici, Pediococcus cerevisiae, Bifidobacterium bifidus; Bifidobacterium longum, Brevibacterium linens, Propionibacterium shermanii; Propionibacterium arabinosum. Penicillium roquefortii, Penicillium camembertii and Staphylococcus xylosus.

38. A method for supplementing the diet of an animal in need thereof comprising orally administering to the animal the formulation according to claim 32 in a regimen of about 0.001 grams to about 100 grams/70 kg body weight/day.

39. A method for supplementing the diet of an animal in need thereof comprising orally administering to the animal the formulation according to claim 37 in a regimen of about 0.25 grams to about 50 grams/70 kg body weight/day.
